(19) United States
(12) Patent Application Publication
(10) Pub. No.: US 2006/0259048 A1
(43) Pub. Date: Nov. 16, 2006

(54) Blunt intercostal suture needle

(76) Inventor: Tomoaki Koseki, Chiyoda-ku (JP)

Correspondence Address: Tomoaki Koseki, 2-17-2 Sotokanda, Chiyoda-ku, 101-0021, JP

(21) Appl. No.: 11/435,047

(22) Filed: May 15, 2006

(30) Foreign Application Priority Data
May 16, 2005 (JP) JP2005-003257 U

(51) Int. Cl. A61B 17/04

(52) U.S. Cl. 606/148

(57) ABSTRACT

When closing the sternum in cardiothoracic surgery, elderly people with a fragile sternum can experience bleeding from the suture sites because of loose knots. The knots are loose because of the danger of cutting into the sternum from the tension caused by the suture wire. In extreme cases, the sternum can be fractured. If the closure is attempted intercostally, there are internal thoracic arteries that run longitudinally underneath the sternum and they may be hurt if a sharp needle is used. Intercostal fixation on its own is inadequate, and additional wires that directly insert into the manubrium are required. Of the sternal closure wires currently available, there is discordance in the numbers provided and actually used, resulting in leftovers that need unnecessary sterilization and reuse. To solve the above problems, an intercostal specific blunt needle has been devised, and packaged together with a sharp needle that will directly insert into the sternum. The set may contain one of each needle or a required combination of both, and come in a pre-sterilized pack. The needle is blunt at the cutting edge, and has a hook, or a side hole. The opposite end has a handle attached. The needle is configured so the wire can be hooked on, or threaded through the hook and hole respectively, and the operative procedure involves lifting up the wires.

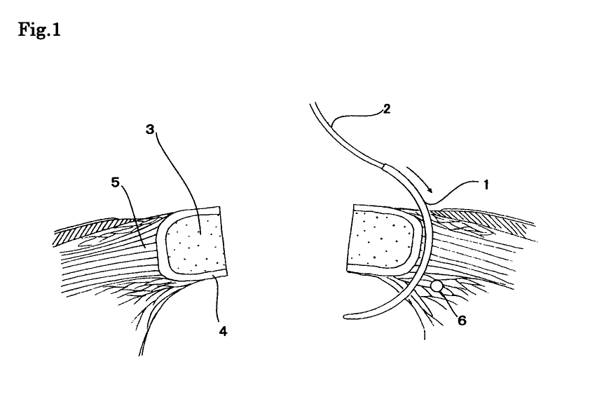

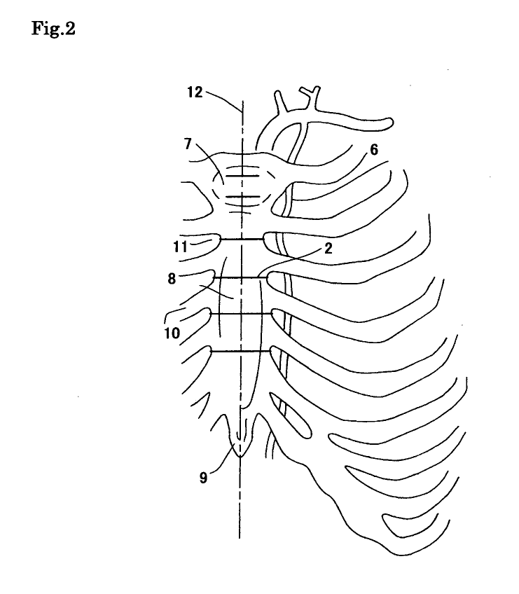

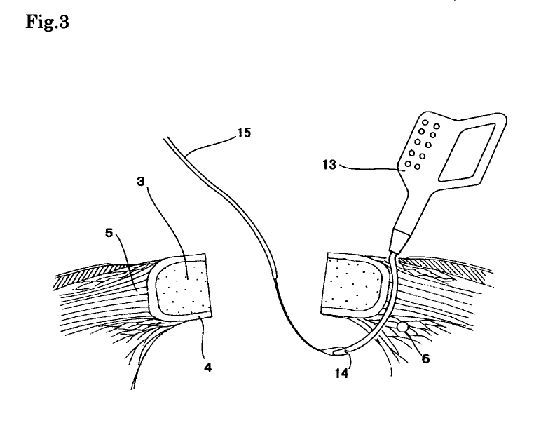

### CROSS-REFERENCE TO RELATED APPLICATIONS

Not Applicable

### STATEMENT REGARDING FEDERALLY SPONSORED RESEARCH OR DEVELOPMENT

Not Applicable

### INCORPORATION-BY-REFERENCE OF MATERIAL SUBMITTED ON A COMPACT DISC or REFERENCE TO A “MICROFICHE APPENDIX”

Not Applicable

### BACKGROUND OF THE INVENTION

(1) Field of the Invention

The invention concerns the suture needle involved in the closure of the sternum in cardiac surgery.

(2) Description of Related Art Including Information Disclosed Under 37 CFR 1.97 and 1.98

The suture needle involved in the closure of the sternum can be differentiated into two types. The first has a wire directly connected behind the sharp end of the needle. The other type uses a wire which is tapered at either end and threaded into the tip of a sharp needle that has penetrated the sternum, and there the wire is lifted up. Patent document 1, diagram 3, demonstrates a suturing method where a drill is used to open the sternum, and a looped suture is lifted up using a hook. Patent documents 2 and 3 have combined the above approaches, and have suggested attaching a sharp needle to the tapered end of the wire. It is currently conventional to use the type where the wire is directly connected to the end of the needle.

Patent Document 1

Patent publication number 2001-198131 (P2001-198131A)

Patent Document 2

Patent publication nubmer 2002-224122 (P2002-224122A)

Patent Document 3

Patent publication number 2003-79632 (P2003-79632A)

### BRIEF SUMMARY OF THE INVENTION

The sternum is a delicate tissue that is composed of cancellous bone in the centre. Cancellous bone is rich in blood, and the direct penetration with a sharp needle will cause it to bleed, resulting in difficulties in its hemostasis.

In elderly people with fragile bones, the cancellous bone of the sternum is surrounded by only a thin outer layer of cortical bone. After directly penetrating the sternum with a sharp needle, the stainless steel wire which is attached to the needle will be tightened over the sternum. This can cause a wire cutting phenomenon to occur where the wire cuts into the bone. Not only will this result in inadequate tightening of the suture knots, but it will also cause bleeding from the suture sites. In extreme cases, fractures of the sternum can occur.

If the wire is inserted through intercostally, it has the advantage that only the cortical bone is in contact with the wire, and therefore wire cutting can be prevented. However, if a sharp needle is used for the intercostal suture, there is the danger of injuring the internal thoracic arteries that run vertically down the sternum.

Fixation of the sternum is inadequate with intercostal sutures on its own; therefore it must be reinforced by wires sutured with a sharp needle directly through the manubrium, a bone forming the top end of the sternum.

For the closure of the sternum, it is standard for Japanese medical establishments to use 5 or 6 sharp needles and wires. However, in most sternal suture sets, there is only one wire and needle in a set or it comes in sets of four. Therefore, unnecessary sutures are leftover after surgery, and medical establishments are forced to sterilize and reuse the remainders. Opening the set with only one wire and needle is impractical due to the amount of labor involved.

To penetrate through the intercostal soft tissue, the needle used is not made sharp, but blunt at the cutting edge. Because the blunt needle which is inserted intercostally, and the sharp needle inserted directly through the sternum are used at the same time, a set of these needles can be composed of one of each needle or a required combination of both. The set will come in pre-sterilized packs.

The needle is set up so that it has a hook or a side hole near the cutting edge. The opposite end has a handle attached. For the closure of the sternum using our technique, the operative procedure firstly involves suturing from above the sternum, and completely through the intercostal soft tissue. The needle is configured so a wire can be hooked on, or threaded through the hook or side hole respectively. After the attachment is made, the wire is lifting up. The blunt intercostal suture needle with a handle attached, and the sharp sternal suture needle with a handle attached, which is directly used to penetrate through the sternum, are paired in a set, or a required combination of both. The set will come in pre-sterilized packs.

### BRIEF DESCRIPTION OF THE SEVERAL VIEWS OF THE DRAWINGS

Diagram 1: Explains how to use the blunt intercostal needle.

Diagram 2: Shows the positions of the sternum, ribs and tying wire in relation to one another.

Diagram 3: Explains how to use the blunt intercostal suture needle with a handle attached.

### DETAILED DESCRIPTION OF THE INVENTION

As described above, the intercostal tissue can be adequately penetrated by using a blunt needle; therefore our idea is that it will be unnecessary to use a sharp needle. This has the benefits of reducing trauma and providing a safe method of closing the sternum, as the likelihood of damaging internal thoracic arteries and nearby tissue is minimal. Because it does not cause wire cutting, it prevents the occurrence of bleeding and fractures. This alleviates the need for hemostasis and has the effect of shortening the total operating time. The sharp needles have been included in the same pre-sterilized packs. Therefore from opening a single pack, it is now possible to have the desired combination of both the needles. The sharp needle is used for the closure of the manubrium which is located at the top end of the sternum, and the blunt intercostal needle is used for suturing of the bottom part. It is now possible to avoid unnecessary sterilization and disposal of the needles by the new way of packaging.

The intercostal specific blunt suture needle reduces tissue trauma and operating time, and by combining it with the sharp needle, the sets have eliminated unnecessary labor.

Diagram 1 shows an example of implementing our idea and shows a cross sectional diagram of the blunt intercostal suture needle (1) in use registered under section 1 of our claim. The sternum is structured in a way that the spongy cancellous (3) bone, which contains a lot of blood, is surrounded by cortical bone (4). In cardiothoracic surgery, the sternum is split longitudinally down the center, and to close this incision, the standard procedure is to directly suture into the sternum on either side of the incision, and close by aligning the bone. The cortical bone (4) forms a thin layer above and below the bone. Therefore in elderly people with fragile bones, if the sternal incision is closed too tightly, the bone may not withstand the tension of the wire. This results in the bone cutting open. As opposed to this, if the suture needle is passed through intercostally, so that it surrounds the outside of the sternum, the wire will only contact the hard cortical bone (4). Therefore even with strong traction, the bone will not easily fracture. However, there is the internal thoracic artery (6) that runs underneath the sternum, and if it is cut accidentally with the needle tip, it will cause significant bleeding. This problem can be avoided by rounding the needle tip and making the needle blunt, allowing for the smooth passage through the soft tissue. As seen in the diagram, the blunt intercostal needle (1) is manufactured curved, and the cutting end is blunt, therefore the risk of damaging the internal thoracic artery (6) is minimal. The blunt intercostal needle (1) has a wire (2) connected at the non-cutting end. After suturing from top to bottom, the opposite side is sutured from bottom to top, the needle is cut, and the wire (2) is tied above the sternum.

Diagram 2 shows the arrangement of the bones and wire tying positions when looking at the human body from in front. The sternum is located approximately at the level of the Adam's apple and extends to the solar plexus. It is bound in front by the ribs. There are three parts that make up the sternum. From the top; the manubrium, the body of the sternum, and the xiphisternum. In approaching the heart, the sternum is cut (incision line 12) longitudinally in the middle. The ribs connect on either side of the sternum. The area in between the ribs longitudinally is called the intercostal region, and contains soft tissues such as muscles and fat. To avoid vertical misalignment after suturing, wires are directly penetrated through the manubrium for closure. In the diagram, there are two wires directly inserted into the manubrium and the four intercostal wires closing the body of the sternum.

Diagram 3 shows the blunt needle (13) with a resin-based handle molded and inserted into the end of the needle, and concerns section 3 and 4 of our claim. The blunt needle is curved and there is a hook (14) at the cutting end. The needle is set up so that a wire that is looped at the end (15) can be hooked onto it and lifted up.

Due to a rise in the elderly population and change in dietary habits, the number of cardiothoracic patients is steadily increasing. Concurrently, of the cardiothoracic patients, there are an increasing number of elderly patients with fragile bones. During the closure of the sternum, the fragility of their bones causes the wires to cut into their bone, a problem called wire cutting. Intercostal suturing has the risk of injuring the internal thoracic arteries. But by using blunt intercostal needles, the wound can be closed safely without the risk of injuring the internal thoracic arteries. As the bleeding is minimal, and the time to achieve hemostasis is short, this leads to a reduction in operating time. Furthermore, because the intercostal suturing is done through soft tissue, it can be done easily and quickly. By combining this with a sharp needle, it is possible for the suturing to be done by opening only one packet. This reduces the work of the theatre nurse, and alleviates the need for sterilizing leftover wires.

1. A suture needle used for the suturing of the sternum in cardiothoracic surgery. The needle, a blunt intercostal suture needle, is used for the passage through the soft tissue located between the ribs, and has the characteristic of being blunt as opposed to being sharp.

2. The blunt intercostal suture needle from section 1 of our claim is combined in a set with a sharp sternal suturing needle, which is directly used to penetrate through the sternum. The set may contain one of each needle or a required combination of both, and come in a pre-sterilized pack to form a suture needle set for sternal closure.

3. The blunt intercostal suture needle from section 1 of our claim has a hook, or a side hole on the cutting end and a handle attached at the opposite end of the needle. After passing this needle through the intercostal soft tissue, a wire is hooked onto the hook, or threaded through the hole at the cutting edge of the needle, so that the procedure for sternal closure is done by lifting up the wire.

4. The blunt intercostal suture needle with a handle from section 3 of our claim is combined in a set with a sharp sternal suturing needle, which is directly used to penetrate through the sternum. The set may contain one of each needle or a required combination of both, and come in a pre-sterilized pack to form a suture needle set for sternal closure.
